(19) United States
(12) Patent Application Publication
(10) Pub. No.: US 2024/0288373 A1
(43) Pub. Date: Aug. 29, 2024

(54) LIGHT INTENSITY MEASUREMENT SYSTEM, RAMAN SCATTERED LIGHT SPECTROSCOPIC MEASUREMENT SYSTEM, AND LIGHT INTENSITY MEASUREMENT METHOD

(71) Applicants: SEED CO., LTD., Bunkyo-ku, Tokyo (JP); KEIO UNIVERSITY, Minato-ku, Tokyo (JP)

(72) Inventors: Taku Kinoshita, Bunkyo-ku, Tokyo (JP); Mio Sawada, Bunkyo-ku, Tokyo (JP); Ari Shinojima, Shinjuku-ku, Tokyo (JP); Toshihide Kurihara, Shinjuku-ku, Tokyo (JP)

(73) Assignees: SEED CO., LTD., Bunkyo-ku, Tokyo (JP); KEIO UNIVERSITY, Minato-ku, Tokyo (JP)

(21) Appl. No.: 18/573,613

(22) PCT Filed: Jul. 11, 2022
(86) PCT No.: PCT/JP2022/027303

§ 371 Date: Dec. 22, 2023

(30) Foreign Application Priority Data
Jul. 14, 2021 (JP) 2021-116644

(51) Int. Cl. G01N 21/65; A61B 3/117; G01N 21/47

(52) U.S. Cl. CPC G01N 21/65; A61B 3/117; G01N 21/47; G01N 2021/4726; G01N 2201/0612; G01N 2201/0683

(57) ABSTRACT

A light intensity measurement system includes a light source unit configured to emit coherent light to aqueous humor of a subject eye, a polarization control unit that is arranged between the light source unit and the subject eye, and configured to control a polarization state of the coherent light, and a measurement unit configured to measure light intensity of scattered light in the aqueous humor of the coherent light. With this configuration, a light intensity measurement system that can non-invasively measure the concentration of substances contained in aqueous humor of a subject eye is provided.

### CROSS REFERENCE TO RELATED APPLICATIONS

This is the U.S. National Phase application of PCT/JP2022/027303, filed Jul. 11, 2022, which claims priority to Japanese Patent Application No. 2021-116644, filed Jul. 14, 2021, the disclosures of these applications being incorporated herein by reference in their entireties for all purposes.

### FIELD OF THE INVENTION

The present invention relates to a light intensity measurement system, a Raman scattered light spectroscopic measurement system, and a light intensity measurement method.

### BACKGROUND OF THE INVENTION

There has been conventionally known a technique of analyzing substances contained in aqueous humor in a subject eye to assist in diagnosis of the subject eye. Patent Literature 1 discloses a technique of non-invasively measuring, using light, substances contained in aqueous humor of a subject eye, to ease the burden on a subject. This technique guides polarization-controlled light into aqueous humor via a cornea, and directly detects light that comes out again via the cornea. Then, by measuring a polarization state of the detected light, the technique measures concentrations of various types of substances contained in the aqueous humor.

On the other hand, in the case of guiding light into aqueous humor via a cornea, the polarization state of light is influenced by the birefringence of the cornea when the light is guided into the aqueous humor, and when the light comes out from the aqueous humor. Thus, to measure the concentration of substances contained in the aqueous humor, it is necessary to exclude the influence exerted by the birefringence of the cornea, from a measurement result. Patent Literature 1 discloses a technique of compensating for the influence exerted by the birefringence of the cornea, using a polarization plate.

### PATENT LITERATURE

Patent Literature 1: WO 2005/067522 A
Patent Literature 2: U.S. Pat. No. 6,885,882

### SUMMARY OF THE INVENTION

Nevertheless, according to a result of anatomical research on the shape of a corneoscleral limbus that has been conducted by inventors of the present invention, and trial calculation that is based on data on the spreading of light fluxes to be guided, and an eyeball movement, in many subject eyes, to realize an optical path for measuring the concentration of substances in aqueous humor that is described in Patent Literature 1, it is necessary to control an incident angle of light with respect to a subject eye, with an error smaller than 1°, and it is considered to be difficult to realize the optical path.

Aspects of the present invention have been devised in view of the foregoing, and provides a light intensity measurement system, a Raman scattered light spectroscopic measurement system, and a light intensity measurement method that can non-invasively measure the concentration of substances contained in aqueous humor of a subject eye.

To solve the problem described above and to achieve the object, a light intensity measurement system according to aspects of the present invention includes: a light source unit configured to emit coherent light to aqueous humor of a subject eye; a polarization control unit that is arranged between the light source unit and the subject eye, and configured to control a polarization state of the coherent light; and a measurement unit configured to measure light intensity of scattered light of the coherent light in the aqueous humor.

In the light intensity measurement system according to aspects of the present invention, the polarization control unit includes any one of a plurality of wave plates, a Babinet-Soleil compensator, and an acousto-optical element.

The light intensity measurement system according to aspects of the present invention includes: a notch filter configured to selectively block light with a wavelength component of Rayleigh scattered light; and a driving unit configured to insert and remove the notch filter into and from between the subject eye and the measurement unit.

A Raman scattered light spectroscopic measurement system according to aspects of the present invention includes: a light source unit configured to emit coherent light to aqueous humor of a subject eye; a polarization control unit that is arranged between the light source unit and the subject eye, and configured to control a polarization state of the coherent light; and a measurement unit configured to measure a spectrum of Raman scattered light of the coherent light in the aqueous humor.

In the Raman scattered light spectroscopic measurement system according to aspects of the present invention, the polarization control unit includes any one of a plurality of wave plates, a Babinet-Soleil compensator, and an acousto-optical element.

The Raman scattered light spectroscopic measurement system according to aspects of the present invention includes: a notch filter configured to selectively block light with a wavelength component of Rayleigh scattered light; and a driving unit configured to insert and remove the notch filter into and from between the subject eye and the measurement unit.

A light intensity measurement method according to aspects of the present invention includes: a light emission step of emitting coherent light to aqueous humor of a subject eye; a polarization control step of controlling a polarization state of the coherent light and guiding the coherent light into the subject eye; and a measurement step of measuring, by a measurement unit, light intensity of scattered light of the coherent light in the aqueous humor.

In the light intensity measurement method according to aspects of the present invention, the polarization control step includes optically adjusting any one of a plurality of wave plates, a Babinet-Soleil compensator, and an acousto-optical element, which are provided to control a polarization state of the coherent light, to maximize light intensity of Rayleigh scattered light.

The light intensity measurement method according to aspects of the present invention includes a filter insertion step of inserting a notch filter configured to selectively block light with a wavelength component of Rayleigh scattered light, into between the subject eye and the measurement unit, wherein, in the measurement step, the measurement unit measures a spectrum of Raman scattered light of the coherent light in the aqueous humor.

According to aspects of the present invention, it is possible to realize a light intensity measurement system, a Raman scattered light spectroscopic measurement system, and a light intensity measurement method that can non-invasively measure the concentration of substances contained in aqueous humor of a subject eye.

### BRIEF DESCRIPTION OF THE DRAWINGS

FIG. 1 is a schematic diagram illustrating a configuration of a Raman scattered light spectroscopic measurement system according to an embodiment of the present invention.

FIG. 2 is a flowchart illustrating processing to be executed by the Raman scattered light spectroscopic measurement system.

FIG. 3 is a schematic diagram illustrating a configuration of a Raman scattered light spectroscopic measurement system according to Modified Example 1.

FIG. 4 is a schematic diagram illustrating a configuration of a Raman scattered light spectroscopic measurement system according to Modified Example 2.

### DETAILED DESCRIPTION OF EMBODIMENTS OF THE INVENTION

Hereinafter, the details of a light intensity measurement system, a Raman scattered light spectroscopic measurement system, and a light intensity measurement method according to aspects of the present invention will be described based on an embodiment, but these are not intended to limit the present invention.

### EMBODIMENT

### [Configuration of Raman Scattered Light Spectroscopic Measurement System]

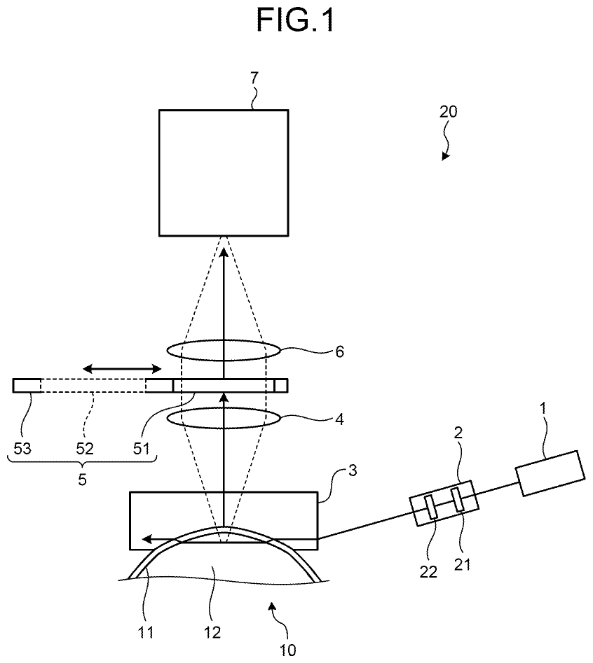

FIG. 1 is a schematic diagram illustrating a configuration of a Raman scattered light spectroscopic measurement system according to an embodiment of the present invention. A Raman scattered light spectroscopic measurement system 20 serving as a light intensity measurement system performs measurement for non-invasively analyzing, by the Raman spectroscopy, the concentration of substances contained in aqueous humor 12 existing inside a cornea 11 of a subject eye 10 being an eye of a subject.

The aqueous humor 12 is tissue fluid filling a chamber sectioned by the cornea 11, an iris, and a crystalline lens existing behind a pupil. The aqueous humor 12 is produced in a ciliary body, circulates inside the chamber via the pupil, and flows out to a vein through a Schlemm's canal from the outside of this chamber. The aqueous humor 12 is used to keep an eye pressure and maintain the shape of an anterior eye chamber of an eyeball. In addition, the aqueous humor 12 has a function of supplying energy by a glucose glycolytic system to eyeball cells such as corneal endothelial cells through which no blood vessel passes.

Although a main component of the aqueous humor 12 is water, not only glucose but also various metabolic products are dissolved in the aqueous humor 12, and it is known that there is correlation between the state of an eye disease and the concentration of a metabolic product. For example, Patent Literature 2 describes that the glucose concentration of the aqueous humor 12 changes after several minutes from a change in blood glucose concentration. There are medical needs such as the recognition of a progress level of retinopathy of diabetes by the measurement of the glucose concentration of the aqueous humor 12, the measurement of cytokine concentration such as a Vascular Endothelial Growth Factor (VEGF), and furthermore, the examination on the medicinal effect of anti-VEGF drug for suppressing the development of a new blood vessel.

The Raman scattered light spectroscopic measurement system 20 includes a light source unit 1, a polarization control unit 2, an eyepiece optical lens 3, a lens 4, a filter 5, a lens 6, and a measurement unit 7.

The light source unit 1 emits coherent light to the aqueous humor 12 of the subject eye 10. The coherent light is used as excitation light for performing spectroscopic measurement of Raman scattered light in the aqueous humor 12. In addition, the wavelength of light emitted by the light source unit 1 is 532 nm, for example, but the wavelength is not specifically limited. The light source unit 1 is only required to be a light source that emits coherent light with a predetermined wavelength, and is a Laser Diode (LD), for example.

The polarization control unit 2 is arranged between the light source unit 1 and the subject eye 10, and controls a polarization state of coherent light. The polarization control unit 2 includes, for example, a λ/4 plate 21 and a λ/2 plate 22. The polarization control unit 2 controls a polarization state of coherent light in such a manner that the light intensity of Rayleigh scattered light in the aqueous humor 12 of coherent light becomes the maximum. Specifically, by rotating high-speed axes and low-speed axes of the λ/4 plate 21 and the λ/2 plate 22 with respect to an optical axis of coherent light, the polarization control unit 2 controls a polarization state of coherent light in such a manner that the light intensity of Rayleigh scattered light becomes the maximum. Note that the polarization control unit 2 may have any configuration as long as a polarization state of coherent light can be controlled, and wave plates are not limited to the λ/4 plate 21 and the λ/2 plate 22, and the polarization control unit 2 may have a configuration including a plurality of wave plates. Furthermore, the polarization control unit 2 may include a Babinet-Soleil compensator or an acousto-optical element. In other words, the polarization control unit 2 is only required to include any one of a plurality of wave plates, a Babinet-Soleil compensator, and an acousto-optical element.

The eyepiece optical lens 3 is an eyepiece optical lens worn by or pushed against the subject eye 10, and guides coherent light emitted from the light source unit 1, into the aqueous humor 12. The eyepiece optical lens 3 may be formed of a material of a soft contact lens system such as Polyhydroxyethylmethacrylate (pHEMA) or silicone hydrogel (SHG), for example, but may be formed of a material of a hard contact lens system.

The lens 4 condenses scattered light from the aqueous humor 12.

The filter 5 includes a notch filter 51, a neutral density filter 52, and a switching plate 53 serving as a driving unit.

The notch filter 51 is a band-stop filter that selectively blocks light with a wavelength component of Rayleigh scattered light. Nevertheless, in the case of measuring Stokes Raman Scattering, in place of the notch filter 51, a filter that lets through light with a wavelength longer than that of coherent light emitted to the aqueous humor 12 may be used. In addition, in the case of measuring anti-Stokes Scattering, in place of the notch filter 51, a filter that lets through light with a wavelength shorter than that of coherent light emitted to the aqueous humor 12 may be used. In addition, in the case of measuring Raman scattered light with a known wavelength, in place of the notch filter 51, a bandpass filter that selectively lets Raman scattered light through may be used.

The neutral density filter 52 lets Rayleigh scattered light from the aqueous humor 12 through while reducing the light intensity of the Rayleigh scattered light. This is to avoid a trouble such as the saturation of the measurement unit 7 by reducing the light intensity of Rayleigh scattered light using the neutral density filter 52 in accordance with the sensitivity of the measurement unit 7, because the measurement unit 7 has the sensitivity suitable for the light intensity of Raman scattered light. Nevertheless, depending on relationship between the light intensity of Rayleigh scattered light and the sensitivity of the measurement unit 7, without using a neutral density filter, Rayleigh scattered light may be let through without being reduced. In other words, without using the neutral density filter 52, a hollow portion may be provided in the switching plate 53.

The switching plate 53 inserts and removes the notch filter 51 into and from between the subject eye 10 and the measurement unit 7.

The lens 6 condenses light having passed through the filter 5, to the measurement unit 7.

The measurement unit 7 measures a spectrum of Raman scattered light of coherent light in the aqueous humor 12. Specifically, the measurement unit 7 includes a spectral device such as a diffractive grating that disperses received light, and a detector that detects the light intensity of dispersed light with each wavelength, such as a charge-coupled device (CCD) sensor arranged in a one-dimensional array. Then, the measurement unit 7 measures light in a direction approximately orthogonal to light emitted by the light source unit 1 to the aqueous humor 12. This is because Raman scattered light can be efficiently measured since the light intensity of Rayleigh scattered light becomes the weakest in a direction approximately orthogonal to excitation light.

### [Raman Scattered Light Spectroscopic Measurement Method Used by Raman Scattered Light Spectroscopic Measurement System]

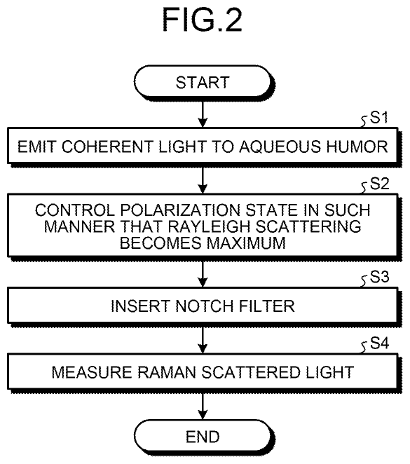

Next, a Raman scattered light spectroscopic measurement method being a light intensity measurement method to be used by the Raman scattered light spectroscopic measurement system 20 will be described. FIG. 2 is a flowchart illustrating processing to be executed by the Raman scattered light spectroscopic measurement system. Note that, in an initial state, the switching plate 53 of the filter 5 inserts the neutral density filter 52 onto an optical path extending from the subject eye 10 toward the measurement unit 7.

First of all, in a state in which the eyepiece optical lens 3 is pushed against the cornea 11 of the subject eye 10, the Raman scattered light spectroscopic measurement system 20 emits coherent light from the light source unit 1 to the aqueous humor 12 of the subject eye 10 from a direction approximately orthogonal to an eye axis of the subject eye 10 (Step S1). At this time, scattered light scattered by substances contained in the aqueous humor 12 travels in an arbitrary direction. In other words, scattered light can be received in all directions. Here, it is preferable that, by adjusting relative positions of the subject eye 10 and the Raman scattered light spectroscopic measurement system 20, the measurement unit 7 is arranged in a direction approximately conforming to the eye axis of the subject eye 10. Because the light intensity of Rayleigh scattered light becomes the weakest by performing such position adjustment, Raman scattered light can be efficiently measured.

Scattered light in the aqueous humor 12 travels from the eyepiece optical lens 3 to the outside, is condensed by the lens 4, is reduced by the neutral density filter 52 of the filter 5 in such a manner as to have the light intensity suitable for the sensitivity of the measurement unit 7, is further condensed by the lens 6, and enters the measurement unit 7. The light guided into the measurement unit 7 is dispersed by the diffractive grating or the like, and detected by the CCD sensor arranged in a one-dimensional array. Accordingly, a spectrum of scattered light in the aqueous humor 12 can be measured.

Subsequently, the polarization control unit 2 controls a polarization state of coherent light emitted from the light source unit 1, in such a manner that the light intensity of Rayleigh scattered light in the aqueous humor 12 becomes the maximum, and guides the coherent light into the subject eye 10 (Step S2). This is based on the fact that, in a polarization state in which the light intensity of Raman scattered light becomes the maximum, the light intensity of Rayleigh scattered light also becomes the maximum. Because Rayleigh scattering has the same wavelength as light emitted from the light source unit 1, it is sufficient that, in the spectrum of scattered light measured by the measurement unit 7, the light intensity of light with the same wavelength as that of light emitted from the light source unit 1 is observed. The polarization control unit 2 rotates high-speed axes and low-speed axes of the λ/4 plate 21 and the λ/2 plate 22 with respect to an optical axis of coherent light in such a manner that the light intensity of Rayleigh scattered light becomes the maximum, in the spectrum measured by the measurement unit 7. At this time, the neutral density filter 52 is arranged on the optical path extending toward the measurement unit 7, and lets part of Rayleigh scattered light through. Note that the control of the polarization control unit 2 may be executed by a control device that automatically rotates the λ/4 plate 21 and the λ/2 plate 22 in tandem with the measurement unit 7, or may be executed by a human while checking a measurement value of the measurement unit 7.

After that, by driving the switching plate 53 of the filter 5 manually or automatically using a motor, and sliding the switching plate 53 approximately vertically to an optical path extending from the subject eye 10 toward the measurement unit 7, the notch filter 51 is inserted onto this optical path (Step S3). By inserting the notch filter 51, it is possible to selectively block Rayleigh scattered light from the aqueous humor 12 that accounts for the large portion of scattered light, and let only remaining Raman scattered light through. Accordingly, it is possible to measure Raman scattered light weaker in light intensity as compared with Rayleigh scattered light.

Furthermore, Raman scattered light in the aqueous humor 12 is received by the measurement unit 7, and a spectrum of Raman scattered light is measured (Step S4). The measurement unit 7 disperses the received light by the diffractive grating, detects dispersed light with each wavelength by the CCD or the like, and measures a spectrum of Raman scattered light. The spectrum of Raman scattered light is converted into an energy difference (Raman shift) between a wavelength of excitation light and each peak wavelength of Raman scattered light. Because this Raman shift has a value corresponding to substances that causes scattering, by comprehensively evaluating the intensity of spectrum and an energy difference, analysis of concentrations of a plurality of types of substances contained in the aqueous humor 12 can be performed.

According to the above-described embodiment, by measuring a spectrum of Raman scattered light in the aqueous humor 12 by the measurement unit 7, it is possible to measure the concentration of substances contained in the aqueous humor 12. It is difficult to realize the optical path in Patent Literature 1 that directly detects light having passed through the aqueous humor 12 of the subject eye 10, but in the embodiment, because Raman scattered light is measured by the measurement unit 7 from a direction approximately orthogonal to light guided into the aqueous humor 12, it is possible to non-invasively measure the concentration of substances contained in the aqueous humor 12 of the subject eye 10.

In addition, because the amplitude of light intensity at a peak of a vibration mode appearing in the spectrum of Raman scattered light depends on a polarization state of light guided into the aqueous humor 12, it is preferable that Raman scattered light is maximized by the polarization control unit 2. Nevertheless, because the light intensity of Raman scattered light is extremely smaller as compared with another type of scattered light such as Rayleigh scattered light, it is difficult to surely capture Raman scattered light and control a polarization state. Furthermore, because the light intensity of light emitted from the light source unit 1 is restricted from the aspect of safety of the subject eye 10, it is impossible to increase the light intensity of light emitted from the light source unit 1, to increase the light intensity of Raman scattered light. Here, to maximize Raman scattered light in the aqueous humor 12, it is sufficient that the polarization state of light guided into the aqueous humor 12 is made into linear polarization vertical to a traveling direction of light. At this time, the light intensity of Rayleigh scattered light also becomes the maximum. Thus, in the embodiment, by maximizing the light intensity of Rayleigh scattered light measured by the measurement unit 7, using the polarization control unit 2, the polarization state of light in the aqueous humor 12 is made into linear polarization vertical to a traveling direction of light, and after that, the notch filter 51 that removes Rayleigh scattered light is inserted. Accordingly, according to the Raman scattered light spectroscopic measurement system 20, it is possible to perform measurement using light in the polarization state that maximizes the light intensity of Raman scattered light.

Note that, in the above-described embodiment, by maximizing Rayleigh scattered light from the aqueous humor 12 using the polarization control unit 2, and making the polarization state of light in the aqueous humor 12 into linear polarization vertical to a traveling direction of light, Raman scattered light is maximized, but the configuration is not limited to this. Making the polarization state of light in the aqueous humor 12 into linear polarization vertical to a traveling direction of light is synonymous with compensating for the influence exerted by the birefringence of the cornea 11. In other words, this corresponds to deriving an axis direction θ and a phase difference δ that constitute the birefringence of the aqueous humor 12. By controlling the polarization control unit 2 using the derived axis direction θ and the phase difference δ, the polarization state of light in the aqueous humor 12 can be controlled to become an arbitrary polarization state.

First of all, in the polarization control unit 2, with respect to a traveling direction of coherent light emitted by the light source unit 1, the λ/4 plate 21 and the λ/2 plate 22 are arranged in this order. If a polarization state of coherent light emitted by the light source unit 1 is represented by a Stokes vector S, and a polarization state caused after the coherent light has passed through the λ/4 plate 21 and the λ/2 plate 22 is represented by a Stokes vector S′, the following formula (1) is obtained. Note that the Stokes vectors S and S′ are four-component column vectors each including zeroth to third components. In a Stokes vector, a zeroth component indicates energy of light, and first to third components indicate polarization states of light.

$${S}^{′} = {M}_{h} {M}_{q} S \qquad ( 1 )$$

In this formula, M_{q} and Mₕ respectively denote Mueller matrices of the λ/4 plate 21 and the λ/2 plate 22, and can be represented by the following formulae (2) and (3). θ_{q} and θₕ respectively denote rotational angles of the λ/4 plate 21 and the λ/2 plate 22 from a horizontal direction.

$${M}_{q} = ( \begin{matrix}1 & 0 & 0 & 0 \\ 0 & {cos}^{2} 2 {θ}_{q} & cos 2 {θ}_{q} sin 2 {θ}_{q} & - sin 2 {θ}_{q} \\ 0 & cos 2 {θ}_{q} sin 2 {θ}_{q} & {sin}^{2} 2 {θ}_{q} & cos 2 {θ}_{q} \\ 0 & sin 2 {θ}_{q} & - cos 2 {θ}_{q} & 0\end{matrix} ) \qquad ( 2 )$$

$${M}_{h} = ( \begin{matrix}1 & 0 & 0 & 0 \\ 0 & cos 4 {θ}_{h} & sin 4 {θ}_{h} & 0 \\ 0 & sin 4 {θ}_{h} & - cos 4 {θ}_{h} & 0 \\ 0 & 0 & 0 & - 1\end{matrix} ) \qquad ( 3 )$$

Here, if coherent light emitted by the light source unit 1 is assumed to be horizontal linearly polarized light, the Stokes vector S of the light can be represented as S=(1100)^{T}. Here, T represents transposition of a matrix element.

In addition, if a Mueller matrix representing the birefringence of a cornea is denoted by M_{b}, using an axis direction θ and a phase difference δ that constitute the birefringence of the cornea, the following formula (4) is obtained.

$${M}_{b} = ( \begin{matrix}1 & 0 & 0 & 0 \\ 0 & {cos}^{2} 2 θ + {sin}^{2} 2 θcosδ & cos 2 θsin 2 θ ( 1 - cos δ ) & - sin 2 θsinδ \\ 0 & cos 2 θsin 2 θ ( 1 - cos δ ) & {sin}^{2} 2 θ + {cos}^{2} 2 θcosδ & cos 2 θsinδ \\ 0 & sin 2 θsinδ & - cos 2 θsinδ & cos δ\end{matrix} ) \qquad ( 4 )$$

Furthermore, a Mueller matrix Mₚₛ of scattering caused when light is scattered in a direction forming 90° with incident light in the aqueous humor 12 is represented by the following formula (5). Note that A denotes a constant serving as a factor in determining the light intensity of scattered light, with respect to excitation light intensity that does not depend on the polarization state.

$${M}_{ps} = A ( \begin{matrix}1 & - 1 & 0 & 0 \\ - 1 & 1 & 0 & 0 \\ 0 & 0 & 0 & 0 \\ 0 & 0 & 0 & 0\end{matrix} ) \qquad ( 5 )$$

Then, because light intensity I of scattered light to be detected by the measurement unit 7 is proportional to the zeroth component of a Stokes vector S″=MₚₛM_{b}MₕM_{g}S, using constants K, L, and M, the light intensity I can be represented by the following formula (6). Note that α is a constant appearing in a phase due to the synthesis of cos and sin, and is unrelated to subsequent optimization for maximizing the light intensity I.

$$I = K cos 2 {θ}_{q} sin ( 4 {θ}_{h} - 2 {θ}_{q} + α ) + L sin 2 {θ}_{q} + M \qquad ( 6 )$$

Note that constants K, L, and M are values defined based on the “axis direction θ and the phase difference δ”. Therefore, by measuring a change in the light intensity I that is caused in a case where the axis direction θ and the phase difference δ are changed by the polarization control unit 2, it is possible to obtain the axis direction θ and the phase difference δ that maximize the light intensity I. In addition, as a method of maximizing the light intensity I, a change in the light intensity I that is caused in a case where the axis direction θ and the phase difference δ are changed may be measured, or the constants K, L, and M may be strictly calculated using the above-described formulae (1) to (6). Calculating the constants K, L, and M is synonymous with calculating the axis direction θ and the phase difference δ, and a polarization state of coherent light in the aqueous humor 12 can be controlled to become an arbitrary polarization state.

### Modified Example 1

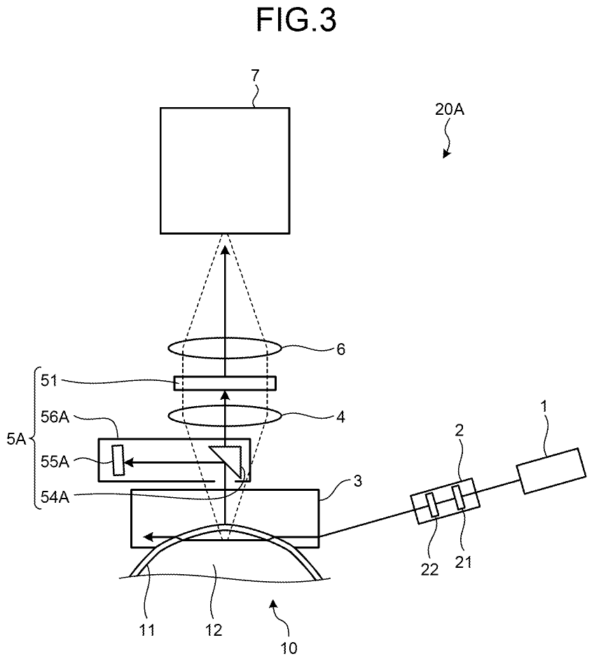

FIG. 3 is a schematic diagram illustrating a configuration of a Raman scattered light spectroscopic measurement system according to Modified Example 1. As illustrated in FIG. 3, an optical branching unit 5A of a Raman scattered light spectroscopic measurement system 20A includes a notch filter 51, a reflecting mirror 54A, a photodetector 55A, and a slide box 56A.

The material of the reflecting mirror 54A is not specifically limited as long as the reflecting mirror 54A is a mirror with high reflectivity, but it is preferable that reflectivity is constant for polarization of reflected light, and includes a metal mirror such as an aluminum, for example. Because light influenced by the birefringence of the cornea 11 enters the reflecting mirror 54A, if reflectivity is not constant for polarization, a polarization state of light in the aqueous humor 12 might fail to be accurately measured.

The photodetector 55A is a photodiode, for example.

The slide box 56A inserts and removes the reflecting mirror 54A onto and from an optical path extending from the aqueous humor 12 toward the measurement unit 7.

Next, a Raman scattered light spectroscopic measurement method to be used by the Raman scattered light spectroscopic measurement system 20A will be described. First of all, Rayleigh scattered light in the aqueous humor 12 is reflected by the reflecting mirror 54A, and detected by the photodetector 55A. By controlling the polarization control unit 2 in this state, the light intensity of Rayleigh scattered light is maximized, and the polarization state of light in the aqueous humor 12 is made into linear polarization vertical to a traveling direction of light. Then, by driving the slide box 56A and removing the reflecting mirror 54A from the optical path extending from the aqueous humor 12 toward the measurement unit 7, Raman scattered light in the aqueous humor 12 enters the measurement unit 7 via the notch filter 51.

According to the Raman scattered light spectroscopic measurement system 20A, by measuring a spectrum of Raman scattered light in the aqueous humor 12 by the measurement unit 7 using light in a polarization state that maximizes the light intensity of Raman scattered light, it is possible to non-invasively measure the concentration of substances contained in the aqueous humor 12. Furthermore, because the notch filter 51 is not moved in the Raman scattered light spectroscopic measurement system 20A, it is possible to prevent a cleaning level of an optical system from lowering due to dust or the like in an apparatus when the notch filter 51 moves.

### Modified Example 2

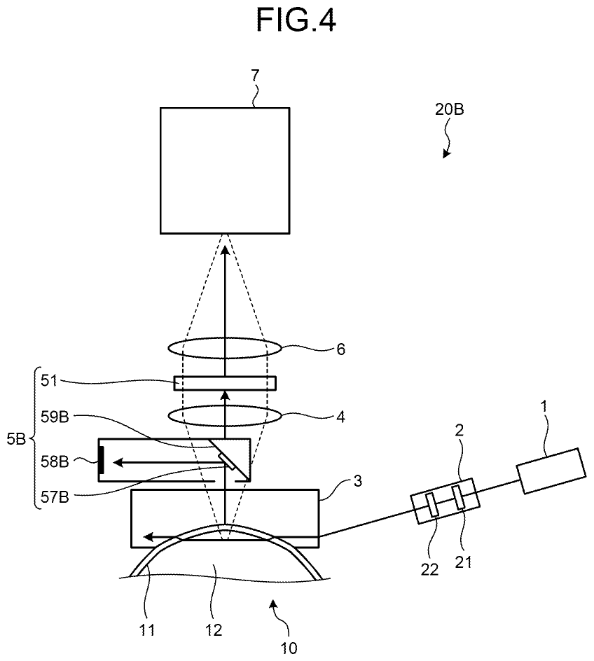

FIG. 4 is a schematic diagram illustrating a configuration of a Raman scattered light spectroscopic measurement system according to Modified Example 2. As illustrated in FIG. 4, a photodetection unit 5B of a Raman scattered light spectroscopic measurement system 20B includes a notch filter 51, a photodetector 57B, a light absorber 58B, and a slide box 59B.

The light absorber 58B absorbs light from the photodetector 57B, and prevents return light from being generated.

The slide box 59B inserts and removes the photodetector 57B onto and from an optical path extending from the aqueous humor 12 toward the measurement unit 7.

Next, a Raman scattered light spectroscopic measurement method to be used by the Raman scattered light spectroscopic measurement system 20B will be described. First of all, Rayleigh scattered light in the aqueous humor 12 is detected by the photodetector 57B. Because the photodetector 57B prevents return light returning to the subject eye 10 from the surface of the photodetector 57B, from being generated, the photodetector 57B is arranged in such a manner as to form 45°, for example, with respect to the optical axis of the subject eye 10. At this time, light reflected on the surface of the photodetector 57B is absorbed by the light absorber 58B. By controlling the polarization control unit 2 in this state, the light intensity of Rayleigh scattered light is maximized, and the polarization state of light in the aqueous humor 12 is made into linear polarization vertical to a traveling direction of light. Then, by driving the slide box 59B and removing the photodetector 57B from the optical path extending from the aqueous humor 12 toward the measurement unit 7, Raman scattered light in the aqueous humor 12 enters the measurement unit 7 via the notch filter 51.

According to the Raman scattered light spectroscopic measurement system 20B, by measuring a spectrum of Raman scattered light in the aqueous humor 12 by the measurement unit 7 using light in a polarization state that maximizes the light intensity of Raman scattered light, it is possible to non-invasively measure the concentration of substances contained in the aqueous humor 12. Furthermore, because the notch filter 51 is not moved in the Raman scattered light spectroscopic measurement system 20B, it is possible to prevent a cleaning level of an optical system from lowering due to dust or the like in an apparatus when the notch filter 51 moves. In addition, in a case where the reflecting mirror 54A is used, even if a metal mirror is used, reflectivity does not always become constant for polarization in some cases, and a measurement result might receive the influence thereof, but a reflecting mirror is not used in the Raman scattered light spectroscopic measurement system 20B. Thus, it is possible to perform measurement without receiving the influence attributed to a reflecting mirror.

In the embodiment, Modified Example 1, and Modified Example 2, the description has been given of an example in which, when a polarization state of coherent light emitted by the light source unit 1 is controlled by the polarization control unit 2, the notch filter 51 is not arranged on an optical path extending from the aqueous humor 12 toward the measurement unit 7 or the photodetector 55A or 57B, and when measurement of Raman scattered light is performed, the notch filter 51 is arranged on an optical path extending from the aqueous humor 12 toward the measurement unit 7, but the example is not limited to this. Any configuration may be employed as long as, when a polarization state of light emitted by the light source unit 1 is controlled by the polarization control unit 2, the notch filter 51 is not arranged on an optical path extending from the aqueous humor 12 toward the measurement unit 7 or the photodetector 55A or 57B, and when measurement of Raman scattered light is performed, the notch filter 51 is arranged on an optical path extending from the aqueous humor 12 toward the measurement unit 7. For example, when a polarization state of light emitted by the light source unit 1 is controlled by the polarization control unit 2, a medical service worker may remove the notch filter 51 from an optical path extending from the aqueous humor 12 toward the measurement unit 7, and when measurement of Raman scattered light is performed, a medical service worker may insert the notch filter 51 onto this optical path.

In addition, in the above-described embodiment, an example of measuring a spectrum of Raman scattered light using the Raman scattered light spectroscopic measurement system 20 has been described, but the example is not limited to this. For example, as in Patent Literature 1, a polarization state of light having passed through the aqueous humor 12 may be measured. In this case, by measuring the light intensity of Rayleigh scattered light, the axis direction θ and the phase difference δ are obtained, and by controlling the polarization control unit 2 in accordance with birefringence of the cornea 11, it becomes possible to control a polarization state of light in the aqueous humor 12 to become an arbitrary polarization state.

### REFERENCE SIGNS LIST

1 LIGHT SOURCE UNIT
2 POLARIZATION CONTROL UNIT
3 EYEPIECE OPTICAL LENS
4, 6 LENS
5 FILTER
5A OPTICAL BRANCHING UNIT
5B PHOTODETECTION UNIT
7 MEASUREMENT UNIT
10 SUBJECT EYE
11 CORNEA
12 AQUEOUS HUMOR
20, 20A, 20B RAMAN SCATTERED LIGHT SPECTROSCOPIC MEASUREMENT SYSTEM
21 λ/4 PLATE
22 λ/2 PLATE
51 NOTCH FILTER
52 NEUTRAL DENSITY FILTER
53 SWITCHING PLATE
54A REFLECTING MIRROR
55A, 57B PHOTODETECTOR
56A, 59B SLIDE BOX
58B LIGHT ABSORBER

1. A light intensity measurement system comprising:
a light source unit configured to emit coherent light to aqueous humor of a subject eye;
a polarization control unit that is arranged between the light source unit and the subject eye, and configured to control a polarization state of the coherent light; and
a measurement unit configured to measure light intensity of scattered light of the coherent light in the aqueous humor.

2. The light intensity measurement system according to claim 1, wherein the polarization control unit includes any one of a plurality of wave plates, a Babinet-Soleil compensator, and an acousto-optical element.

3. The light intensity measurement system according to claim 1, comprising:
a notch filter configured to selectively block light with a wavelength component of Rayleigh scattered light; and
a driving unit configured to insert and remove the notch filter into and from between the subject eye and the measurement unit.

4. A Raman scattered light spectroscopic measurement system comprising:
a light source unit configured to emit coherent light to aqueous humor of a subject eye;
a polarization control unit that is arranged between the light source unit and the subject eye, and configured to control a polarization state of the coherent light; and
a measurement unit configured to measure a spectrum of Raman scattered light of the coherent light in the aqueous humor.

5. The Raman scattered light spectroscopic measurement system according to claim 4, wherein the polarization control unit includes any one of a plurality of wave plates, a Babinet-Soleil compensator, and an acousto-optical element.

6. The Raman scattered light spectroscopic measurement system according to claim 4, comprising:
a notch filter configured to selectively block light with a wavelength component of Rayleigh scattered light; and
a driving unit configured to insert and remove the notch filter into and from between the subject eye and the measurement unit.

7. A light intensity measurement method comprising:
a light emission step of emitting coherent light to aqueous humor of a subject eye;
a polarization control step of controlling a polarization state of the coherent light and guiding the coherent light into the subject eye; and
a measurement step of measuring, by a measurement unit, light intensity of scattered light of the coherent light in the aqueous humor.

8. The light intensity measurement method according to claim 7, wherein the polarization control step includes optically adjusting any one of a plurality of wave plates, a Babinet-Soleil compensator, and an acousto-optical element, which are provided to control a polarization state of the coherent light, to maximize light intensity of Rayleigh scattered light.

9. The light intensity measurement method according to claim 7, comprising:
a filter insertion step of inserting a notch filter configured to selectively block light with a wavelength component of Rayleigh scattered light, into between the subject eye and the measurement unit,
wherein, in the measurement step, the measurement unit measures a spectrum of Raman scattered light of the coherent light in the aqueous humor.
